(19) United States
(12) Patent Application Publication
(10) Pub. No.: US 2008/0194845 A1
(43) Pub. Date: Aug. 14, 2008

(54) Integrated Process For Preparing Trioxane From Formaldehyde

(75) Inventors: Neven Lang, Mannheim (DE); Eckhard Stroefer, Mannheim (DE); Achim Stammer, Freinsheim (DE); Thorsten Friese, Mannheim (DE); Markus Siegert, Heidelberg (DE); Hans Hasse, Kaisershlautern (DE); Thomas Grutzner, Brig (CH); Sergej Blagov, Mainz (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 11/997,619

(22) PCT Filed: Jul. 19, 2006
(86) PCT No.: PCT/EP2006/064406

§ 371 Date: Apr. 4, 2008

(30) Foreign Application Priority Data
Aug. 3, 2005 (DE) 10 2005 036 544.2

(51) Int. Cl. C07D 323/06

(52) U.S. Cl. 549/368

(57) ABSTRACT

The invention relates to an integrated process for preparing trioxane from formaldehyde, comprising the steps of:
a) a stream A1 comprising water and formaldehyde and a recycle stream B2 consisting substantially of water and formaldehyde are fed to a trioxane synthesis reactor and allowed to react to obtain a product stream A2 comprising trioxane, water and formaldehyde;
b) stream A2 is fed to a first low-pressure distillation column and distilled at a pressure of from 0.1 to 2.5 bar to obtain a stream B1 enriched in trioxane and additionally comprising water and formaldehyde, and the recycle stream B2 consisting substantially of formaldehyde and water;
c) stream B1 and a recycle stream D1 comprising trioxane, water and formaldehyde are fed to a second low-pressure distillation column and distilled at a pressure of from 0.1 to 2.5 bar to obtain a stream C1 comprising predominantly trioxane and additionally formaldehyde and water, and a stream C2 consisting substantially of formaldehyde and water;
d) stream C1 is fed to a high-pressure distillation column and distilled at a pressure of from 0.2 to 17.5 bar to obtain the recycle stream D1 and a product stream D2 consisting substantially of trioxane,
in which a removal of the low boilers present in streams B1 or C1 may be undertaken between steps b) and c) or c) and d).

The invention relates to an integrated process for preparing trioxane from formaldehyde.

Trioxane is generally prepared by reactive distillation of aqueous formaldehyde solution in the presence of acidic catalysts. This affords a mixture comprising trioxane, formaldehyde and water as distillate. The trioxane is subsequently extracted from this mixture by extraction with halogenated hydrocarbons such as methylene chloride or 1,2-dichloroethane, or other water-immiscible solvents.

DE-A 1 668 867 describes a process for removing trioxane from mixtures comprising water, formaldehyde and trioxane by extraction with an organic solvent. In this process, an extraction zone consisting of two subzones is charged at one end with an organic, virtually water-immiscible extractant for trioxane, and at the other end with water. Between the two subzones, the distillate from the trioxane synthesis to be separated is fed. On the side of the solvent feed, an aqueous formaldehyde solution is then obtained, and, on the side of the water feed, a virtually formaldehyde-free solution of trioxane in the organic solvent.

A disadvantage of this procedure is the occurrence of extractant which has to be purified. Some of the extractants used are hazardous substances (T or T+ substances in the context of the German Hazardous Substances Directive), whose handling entails special precautions.

DE-A 197 32 291 describes a process for removing trioxane from an aqueous mixture which consists substantially of trioxane, water and formaldehyde, by removing trioxane from the mixture by pervaporation and separating the trioxane-enriched permeate by rectification into pure trioxane on the one hand and an azeotropic mixture of trioxane, water and formaldehyde on the other. In one example, an aqueous mixture consisting of 40% by weight of trioxane, 40% by weight of water and 20% by weight of formaldehyde is separated in a first distillation column under standard pressure into a water/formaldehyde mixture and into an azeotropic trioxane/water/formaldehyde mixture. The azeotropic mixture is passed into a pervaporation unit which comprises a membrane composed of polydimethylsiloxane with a hydrophobic zeolite. The trioxane-enriched mixture is separated in a second distillation column under standard pressure into trioxane and, in turn, into an azeotropic mixture of trioxane, water and formaldehyde. This azeotropic mixture is recycled upstream of the pervaporation stage.

This procedure is very costly and inconvenient. The pervaporation unit in particular entails high capital costs.

It is an object of the invention to provide an alternative process for preparing trioxane from aqueous formaldehyde solution to obtain pure trioxane. It is a particular object to provide a process which avoids the performance of extraction steps or pervaporation steps for obtaining pure trioxane.

The object is achieved by an integrated process for preparing trioxane from formaldehyde, comprising the steps of:
a) a stream A1 comprising water and formaldehyde and a recycle stream B2 consisting substantially of water and formaldehyde are fed to a trioxane synthesis reactor and allowed to react to obtain a product stream A2 comprising trioxane, water and formaldehyde;
b) stream A2 is fed to a first low-pressure distillation column and distilled at a pressure of from 0.1 to 2.5 bar to obtain a stream B1 enriched in trioxane and additionally comprising water and formaldehyde, and the recycle stream B2 consisting substantially of formaldehyde and water;
c) stream B1 and a recycle stream D1 comprising trioxane, water and formaldehyde are fed to a second low-pressure distillation column and distilled at a pressure of from 0.1 to 2.5 bar to obtain a stream C1 comprising predominantly trioxane and additionally formaldehyde and water, and a stream C2 consisting substantially of formaldehyde and water;
d) stream C1 is fed to a high-pressure distillation column and distilled at a pressure of from 0.2 to 17.5 bar to obtain the recycle stream D1 and a product stream D2 consisting substantially of trioxane.

It is known that trioxane, formaldehyde and water form a ternary azeotrope which, at a pressure of 1 bar, consists of 69% by weight of trioxane, 5% by weight of formaldehyde and 26% by weight of water.

According to the invention, the ternary azeotrope is separated by a pressure swing distillation, by carrying out a first and a second distillation stage at different pressures. In a first distillation stage which is operated at low pressure, the starting mixture is separated into a trioxane-rich trioxane/water/formaldehyde mixture with low formaldehyde content on the one hand and a substantially trioxane-free formaldehyde/water mixture on the other. The trioxane-rich trioxane/water/formaldehyde mixture is subsequently separated in a second distillation stage which is carried out at high pressure into a trioxane-rich trioxane/water/formaldehyde mixture on the one hand and pure trioxane on the other. According to the invention, the first distillation stage is carried out in two (low-pressure) distillation columns connected in series. The trioxane-rich mixture from the first low-pressure distillation column and the trioxane-rich mixture from the high-pressure distillation column are distilled in a (middle) second low-pressure distillation column to remove further, substantially trioxane-free formaldehyde/water mixture. This affords high trioxane enrichment.

Useful high-pressure and low-pressure distillation columns are any distillation columns such as columns with structured packing or tray columns. The distillation columns may comprise any internals, structured packings or random packings. In the following, all pressure data relate to the pressure at the top of the column in question.

In a first process step a), a stream A1 comprising water and formaldehyde and a recycle stream B2 consisting substantially of water and formaldehyde are fed to a trioxane synthesis reactor and allowed to react to obtain a product stream A2 comprising trioxane, water and formaldehyde.

In general, stream A1 comprises from 50 to 85% by weight of formaldehyde and from 15 to 50% by weight of water.

Product stream A2 comprises generally from 35 to 84% by weight of formaldehyde, from 15 to 45% by weight of water and from 1 to 30% by weight of trioxane.

In one embodiment of the process according to the invention, the water/formaldehyde mixture is reacted in the trioxane synthesis stage a) in the presence of acidic homogeneous or heterogeneous catalysts such as ion exchange resins, zeolites, sulfuric acid or p-toluenesulfonic acid at a temperature of generally from 70 to 130° C. It is possible to work in a reactive distillation column or a reactive evaporator. The product mixture composed of trioxane, formaldehyde and water is then obtained as a vaporous vapor draw stream of the reactive evaporator or as a top draw stream of the reaction column. The trioxane synthesis may also be carried out in a fixed bed reactor or fluidized bed reactor over a heterogeneous catalyst, for example an ion exchange resin or zeolite.

In a step b) which follows step a), stream A2 is fed to a first low-pressure distillation column and distilled at a pressure of from 0.1 to 2.5 bar to obtain a stream B1 enriched in trioxane and additionally comprising water and formaldehyde, and the recycle stream B2 consisting substantially of formaldehyde and water.

The first low-pressure distillation column comprises preferably from 2 to 50, more preferably from 4 to 40 theoretical plates. In general, the rectifying section of the distillation column comprises at least 25%, preferably from 50 to 90% of the theoretical plates of this distillation column.

The stream B1 enriched in trioxane comprises generally from 35 to 70% by weight of trioxane, from 5 to 20% by weight of formaldehyde and from 10 to 60% by weight of water. Stream B2 comprises generally less than 1% by weight, preferably less than 0.5% by weight of trioxane, more preferably less than 0.1% by weight of trioxane. Recycle stream B2 comprises generally from 20 to 80% by weight of formaldehyde, from 80 to 20% by weight of water and from 0 to 1% by weight of trioxane; it preferably comprises from 30 to 75% by weight of formaldehyde, from 24.9 to 70% by weight of water and from 0 to 0.1% by weight of trioxane.

Preferably, stream B1 is withdrawn as a top draw stream and stream B2 as a bottom draw stream from the first low-pressure distillation column. Stream B1 may also be withdrawn as a side draw stream below the top of the column.

Stream B2 is recycled into the trioxane synthesis stage a).

In one embodiment of the process according to the invention, the trioxane synthesis stage a) and the first low-pressure distillation stage b) are carried out together as a reactive distillation in a reaction column. In the stripping section, this may comprise a fixed catalyst bed of a heterogeneous catalyst. Alternatively, the reactive distillation may also be carried out in the presence of a homogeneous catalyst, in which case an acidic catalyst is present together with the water/formaldehyde mixture in the column bottom.

In a process step c) which follows step b), stream B1 and a recycle stream D1 comprising trioxane, water and formaldehyde are fed to a second low-pressure distillation column and distilled at a pressure of from 0.1 to 2.5 bar to obtain a stream C1 comprising predominantly trioxane and additionally formaldehyde and water, and a stream C2 consisting substantially of formaldehyde and water.

The second low-pressure distillation column comprises generally from 2 to 50, preferably from 10 to 50 theoretical plates. In general, the stripping section of this column comprises at least 25%, preferably from 50 to 90%, of the theoretical plates of this column.

Stream C1 comprises generally more than 50% by weight, preferably more than 60% by weight, more preferably more than 65% by weight of trioxane. For example, stream C2 may comprise from 3 to 20% by weight of formaldehyde, from 10 to 30% by weight of water and from 60 to 80% by weight of trioxane. Stream C2 is substantially trioxane-free, i.e. it comprises less than 1% by weight, preferably less than 0.5% by weight and more preferably less than 0.1% by weight of trioxane. In general, it comprises from 10 to 30% by weight of formaldehyde and from 70 to 90% by weight of water.

The low-pressure distillation columns of stages b) and c) are preferably operated substantially at the same pressure. The pressure difference is generally not more than 1 bar. Stages b) and c) are preferably carried out at a pressure in the range from 0.4 to 1.5 bar.

In principle, streams B1 and D1 may be fed to the second low-pressure distillation column at any point. Preferably, stream B1 is fed as a first side feed and stream D1 as a second side feed to the second low-pressure distillation column, and stream C1 is withdrawn as a top draw stream and stream C2 as a bottom draw stream. Streams B1 and D1 may also be combined and be added as one side feed.

The ratio of streams B1 and D1 is preferably selected such that, overall, a mixture of from 50 to 70% by weight of trioxane, from 5 to 20% by weight of formaldehyde and from 20 to 45% by weight of water is fed to the second low-pressure distillation column.

In a step d) which follows step c), stream C1 is fed to a high-pressure distillation column and distilled at a pressure of from 0.2 to 17.5 bar to obtain the recycle stream D1 and a product stream D2 consisting substantially of trioxane.

In general, the high-pressure distillation column has from 2 to 50 theoretical plates, preferably from 10 to 50 theoretical plates, the stripping section of this distillation column comprising generally from 25 to 90%, preferably from 50 to 75% of the theoretical plates of this column.

In general, product stream D2 comprises from 95 to 100% by weight, preferably from 99 to 100% by weight of trioxane, and from 0 to 5% by weight, preferably from 0 to 1% by weight of water. More preferably, the water content in product stream D2 is <0.1% by weight. It may even be <0.01% by weight. Recycle stream D1 comprises generally from 1 to 15% by weight of formaldehyde, from 10 to 40% by weight of water and from 40 to 65% by weight of trioxane, preferably from 5 to 15% by weight of formaldehyde, from 25 to 40% by weight of water and from 45 to 60% by weight of trioxane.

The pressure in the high-pressure distillation column is at least 0.1 bar higher, but generally at least 0.5 bar higher than in the second low-pressure distillation column. In general, this pressure difference is from 0.5 to 10 bar, preferably from 1 to 7 bar. The high-pressure distillation column of step d) is operated preferably at a pressure in the range from 2.5 to 10 bar.

Preferably, stream C1 is fed as a side feed to the high-pressure distillation column, stream D1 is withdrawn as a top draw stream and stream D2 as a bottom draw stream. Stream D2 may also be withdrawn as a gaseous side draw between feed and column bottom.

In addition to formaldehyde, water and trioxane, streams A2, B1, C1 and D1 in particular may also comprise up to 15% by weight, generally from 1 to 10% by weight of low boilers. Typical low boilers which may be formed in the trioxane synthesis and the subsequent distillative separation are methyl formate, methylal, dimethoxydimethyl ether, methanol, formic acid and also further low-boiling hemiacetals and full acetals. To remove these low boilers, it is optionally possible between the first and the second low-pressure distillation stage, or between the second low-pressure distillation stage and the high-pressure distillation stage, to carry out a low boiler removal stage. In this case, the low boilers are removed preferably via the top of a low boiler removal column which is preferably operated at a pressure of from 1 to 3 bar. In general, the low boiler removal column has at least 5 theoretical plates, preferably from 15 to 50 theoretical plates. The stripping section of this column comprises preferably from 25 to 90% of the theoretical plates of this column. Streams B1 and C1 are fed to this low boiler removal column as a side feed, and the stream B1′ or C1′ freed of the low boilers is generally obtained as a bottom draw stream. When the low boiler removal is carried out, stream B1′ and C1′ are fed as stream B1 and C1 respectively to the downstream second low-pressure distillation column and high-pressure distillation column respectively.

In a preferred embodiment, the process according to the invention additionally comprises steps f) and g). Step f) precedes step a) and step g) follows step e). In step f), feed stream F1 comprising formaldehyde and water and a recycle stream G1 comprising formaldehyde and water are fed to a formaldehyde concentration unit, and stream A1 is withdrawn as a formaldehyde-rich bottom draw stream from the concentration unit. A low-formaldehyde stream F2 is withdrawn as the top or vapor draw stream or bottom draw stream. In a further step g), the formaldehyde-rich recycle stream G1 is obtained from the low-formaldehyde streams C1 and F2. In this step, streams F2 and C2 are fed to a further distillation column and distilled at a pressure of from 1 to 10 bar to obtain the recycle stream G1 and a wastewater stream G2 consisting substantially of water.

The concentration f) of the formaldehyde/water mixture can be carried out in an evaporator or a distillation column; it is preferably carried out in an evaporator. Preferred evaporators are continuous evaporators such as circulation evaporators, falling-film evaporators or thin-film evaporators. A particularly preferred concentration unit is a falling-film evaporator. The falling-film evaporator is operated generally at a pressure of from 50 to 200 mbar and a temperature of from 40 to 75° C.

The concentration step f) can be carried out as described, for example, in DE-A 199 25 870.

The concentration f) of the formaldehyde/water mixture may also be carried out in a pressure distillation column, in which case an aqueous stream which consists substantially of water is drawn off at the column bottom. Such a column may be operated, for example, at a pressure of 5.5 bar, a top temperature of 147° C. and a bottom temperature of 156° C.

The further distillation column of step g) is operated at a pressure in the range from 1 to 10 bar, preferably from 2 to 5 bar. This distillation column has generally from 2 to 50 theoretical plates, preferably from 10 to 50 theoretical plates.

Recycle stream G1 comprises generally from 0 to 1% by weight of trioxane, from 40 to 80% by weight of formaldehyde and from 20 to 60% by weight of water. Stream G2 comprises generally at least 95% by weight, preferably at least 98% by weight and more preferably at least 99% by weight of water.

In general, feed stream F1 is fed to the concentration unit of step f) as a side feed and recycle stream G1 as a top feed.

In general, stream C1 is fed to the further distillation column of step g) as a side feed and stream F2 as a side feed, and recycle stream G1 is withdrawn as a top draw stream and wastewater stream G2 as a bottom draw stream or side draw stream in the stripping section of the column.

In a further preferred embodiment, step h) which precedes step a) is carried out. In this step, a feed stream H1 comprising formaldehyde and water and stream C2 are fed to a formaldehyde concentration unit, stream A1 is obtained as a formaldehyde-rich top or vapor draw stream or else as a side draw stream in the rectifying section of the column, and a wastewater stream H2 consisting substantially of water is obtained as a bottom draw stream.

The concentration of the formaldehyde/water mixture can be carried out in an evaporator or a distillation column; it is preferably carried out in an evaporator.

Preferred evaporators are continuous evaporators such as circulation evaporators, falling-film evaporators, or thin-film evaporators. A particularly preferred concentration unit is a falling-film evaporator. The falling-film evaporator helical-tube evaporators is operated generally at a pressure of from 50 to 200 mbar and a temperature of from 40 to 75° C.

In general, feed stream H1 is fed to the concentration unit as a first side feed and stream C1 as a second side feed below the first side feed.

Top or vapor draw stream A1 comprises preferably from 50 to 70% by weight of formaldehyde and from 30 to 50% by weight of water. Bottom draw stream H2 comprises generally at least 90% by weight, preferably at least 95% by weight and more preferably at least 98% by weight of water.

The invention is illustrated in detail by the examples which follow.

### EXAMPLES

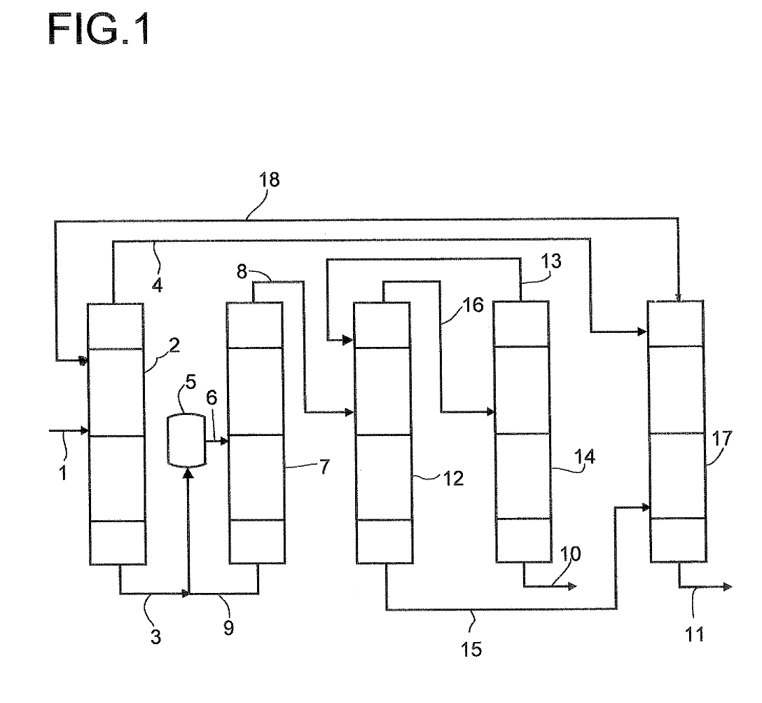

FIG. 1 shows one embodiment of the process according to the invention.

Feed stream 1 composed of 37% by weight of formaldehyde and 63% by weight of water, and recycle stream 18 composed of 57% by weight of formaldehyde and 43% by weight of water are fed to the falling-film evaporator 2. Overall, a mixture of 42% by weight of formaldehyde and 58% by weight of water is thus fed to the falling-film evaporator 2. The falling-film evaporator 2 is operated at a pressure of 0.1 bar and a temperature of 58° C. The vapor draw stream 4 obtained is a mixture of 20% by weight of formaldehyde and 80% by weight of water. The bottom draw stream 3 obtained is a mixture of 72% by weight of formaldehyde and 28% by weight of water. The bottom draw stream 3 is combined with the bottom draw stream 9 of the first low-pressure distillation column 7, and the combined streams are fed to the trioxane synthesis reactor 5 which is configured as a stirred tank. The product stream 6 comprises 68% by weight of formaldehyde, 24% by weight of water and 6% by weight of trioxane. It is fed to the first low-pressure distillation column 7 with 20 theoretical plates at the height of the second theoretical plate. Column 7 is operated at a pressure of 1 bar; the bottom temperature is approx. 105° C., the top temperature approx. 97° C. A top draw stream 8 composed of 8% by weight of formaldehyde, 28% by weight of water and 64% by weight of trioxane, and a bottom draw stream 9 composed of 77% by weight of formaldehyde, 22.7% by weight of water and 0.3% by weight of trioxane are obtained. The top draw stream 8 is fed to the second low-pressure distillation column 12 with 18 theoretical plates at the height of the 7th theoretical plate. In addition, the top draw stream of the high-pressure distillation column 14 composed of 7% by weight of formaldehyde, 29% by weight of water and 64% by weight of trioxane is fed to the column 12 at the height of the 12th theoretical plate. The column 12 is operated at a pressure of 1 bar; the bottom temperature is approx. 102° C., the top temperature approx. 95° C. The top draw stream 16 obtained is a mixture of 6% by weight of formaldehyde, 24% by weight of water and 70% by weight of trioxane. The bottom draw stream 24 obtained is a mixture of 22% by weight of formaldehyde and 78% by weight of water. The top draw stream 16 is fed to the high-pressure distillation column 14 with 32 theoretical plates at the height of the 48th theoretical plate. This column is operated at 5 bar; the bottom temperature is approx. 175° C., the top temperature approx. 140° C. A bottom draw stream 10 comprising more than 99% by weight of trioxane is obtained.

The bottom draw stream 15 of the second low-pressure distillation column is fed to the further column 17 with 32 theoretical plates at the height of the 16th theoretical plate, and the vapor draw stream 4 of the falling-film evaporator 2 at the height of the 16th theoretical plate. This column is likewise operated at a pressure of 5 bar. The bottom temperature is approx. 152° C., the top temperature approx. 138° C. The bottom draw stream 11 comprises 99% by weight of water. The top draw stream 18 comprises 57% by weight of formaldehyde and 43% by weight of water, and is recycled in to the falling-film evaporator 2.

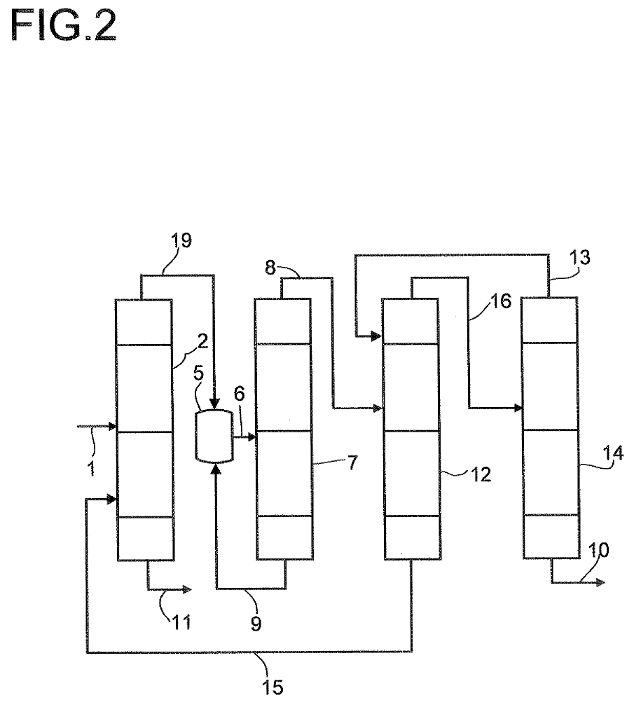

FIG. 2 shows a further embodiment of the process according to the invention.

The feed stream 1 composed of 37% by weight of formaldehyde and 63% by weight of water is fed to the column 2 with 25 theoretical plates at the height of the 15th theoretical plate. Also fed to it at the height of the 10th theoretical plate is the bottom draw stream 15 of the second low-pressure distillation column composed of 23% by weight of formaldehyde and 77% by weight of water. In total, a mixture of 32% by weight of formaldehyde and 68% by weight of water is fed to the distillation column 2 through streams 1 and 15. The column 2 is operated at a pressure of 4 bar The bottom temperature is approx. 144° C., the top temperature approx. 131° C. A bottom draw stream 11 composed of 99% by weight of water, and a top draw stream 19 composed of 57% by weight of formaldehyde and 43% by weight of water are obtained. This stream 19 and the bottom draw stream of the first low-pressure distillation column 7 are fed to the trioxane synthesis reactor 5 which is configured as a fixed bed reactor. A product stream 6 composed of 53% by weight of formaldehyde, 43% by weight of water and 4% by weight of trioxane is obtained. This stream 6 is fed to the low-pressure distillation column 7 with 24 theoretical plates at the height of the 5th theoretical plate. A top draw stream 8 composed of 13% by weight of formaldehyde, 43% by weight of water and 44% by weight of trioxane, and a bottom draw stream 9 composed of 77% by weight of formaldehyde, 22.7% by weight of water and 0.3% by weight of trioxane are obtained. The top draw stream 8 is fed to the second low-pressure distillation column 12 with 32 theoretical plates at the height of the 16th theoretical plate. Also fed to the column 12 at the height of the 24th theoretical plate is the top draw stream 22 of the high-pressure distillation column 23 composed of 10% by weight of formaldehyde, 33% by weight of water and 57% by weight of trioxane. The second low-pressure distillation column 21 is operated at a pressure of 0.8 bar; the bottom temperature is approx. 102° C. and the top temperature approx. 85° C. A bottom draw stream 15 composed of 23% by weight of formaldehyde and 77% by weight of water, and a top stream 16 composed of 6% by weight of formaldehyde, 24% by weight of water and 70% by weight of trioxane are obtained. This stream 16 is fed to the high-pressure distillation column 14 with 28 theoretical plates at the height of the 18th theoretical plate. The column 14 is operated at a pressure of 4 bar; the bottom temperature is approx. 160° C., the top temperature approx. 133° C. The top draw stream 13 which is recycled into the second low-pressure distillation column and a bottom draw stream 10 comprising 99.5% by weight of trioxane are obtained.

1-15. (canceled)

16. An integrated process for preparing trioxane from formaldehyde, comprising:
a) feeding a stream A1 comprising water and formaldehyde and a recycle stream B2 consisting substantially of water, formaldehyde, and less than 1% by weight of trioxane to a trioxane synthesis reactor, wherein said formaldehyde is reacted to form trioxane, and obtaining a product stream A2 comprising trioxane, water, and formaldehyde;
b) feeding said stream A2 to a first low-pressure distillation column and distilling said stream A2 at a pressure of from 0.1 to 2.5 bar to obtain a stream B1 comprising trioxane, water, and formaldehyde, wherein said stream B1 is enriched in trioxane relative to said stream A2, and said recycle stream B2;
c) feeding said stream B1 and a recycle stream D1 comprising trioxane, water, and formaldehyde to a second low-pressure distillation column and distilling said stream B1 and recycle stream D1 at a pressure of from 0.1 to 2.5 bar to obtain a stream C1 comprising trioxane, formaldehyde, and water, and a stream C2 consisting substantially of formaldehyde, water, and less than 1% by weight of trioxane;
d) feeding said stream C1 to a high-pressure distillation column and distilling said stream C1 at a pressure of from 2.5 to 10 bar to obtain said recycle stream D1 and a product stream D2 consisting substantially of trioxane;
wherein removal of the low boilers present in said streams B1 or C1 is optionally performed between after b) and before c) or after c) and before d).

17. The process of claim 16, wherein the low-pressure distillations in b) and c) are carried out at a pressure of from 0.4 to 1.5 bar and the high-pressure distillation in d) is carried out at a pressure of from 2.5 to 7 bar.

18. The process of claim 16, wherein said stream B1 is withdrawn as a top draw stream and said stream B2 is withdrawn as a bottom draw stream from said first low-pressure distillation column.

19. The process of claim 16, wherein said stream B1 is fed as a first side feed and stream D1 is fed as a second side feed to said second low-pressure distillation column, and said stream C1 is withdrawn as a top draw stream and said stream C2 is withdrawn as a bottom draw stream.

20. The process of claim 16, wherein said stream C1 is fed as a side feed to said high-pressure distillation column, said stream D1 is withdrawn as a top draw stream and said stream D2 is withdrawn as a bottom draw stream.

21. The process of claim 16, additionally comprising:
f) feeding a feed stream F1 comprising formaldehyde and water and a recycle stream G1 comprising formaldehyde and water to a formaldehyde concentration unit, withdrawing said stream A1 from said formaldehyde concentration unit as a formaldehyde-rich stream and withdrawing a low-formaldehyde stream F2 from said formaldehyde concentration unit as a bottom, top, or vapor draw stream;
g) feeding said stream F2 and said stream C2 to a further distillation column and distilling said stream F2 and said stream C2 at a pressure of from 1 to 10 bar to obtain said recycle stream G1 and a wastewater stream G2 consisting substantially of water.

22. The process of claim 21, wherein said stream A1 is withdrawn as a formaldehyde-rich bottom draw stream and said stream F2 is withdrawn as a low-formaldehyde top or vapor draw stream.

23. The process of claim 21, wherein said formaldehyde concentration unit is a pressure distillation column and said stream F2 is obtained as an aqueous bottom draw stream.

24. The process of claim 21, wherein said formaldehyde concentration unit is a falling-film evaporator.

25. The process of claim 21, wherein said feed stream F2 and said recycle stream G1 are fed as a side feed to said formaldehyde concentration unit.

26. The process of claim 21, wherein said stream C1 and said stream F2 are fed as a side feed to said further distillation column, said recycle stream G1 is withdrawn as a top draw stream, and said wastewater stream G2 is withdrawn as a bottom draw stream.

27. The process of claim 21, wherein said distillation in g) is performed at a pressure of from 2.5 to 8 bar.

28. The process of claim 16, additionally comprising:
h) feeding a feed stream H1 comprising formaldehyde and water and said stream C2 to a formaldehyde concentration unit, withdrawing stream A1 as a formaldehyde-rich top or vapor draw stream, and withdrawing a wastewater stream H2 consisting substantially of water as a bottom draw stream.

29. The process of claim 28, wherein said formaldehyde concentration unit is a falling-film evaporator.

30. The process of claim 28, wherein said feed stream H1 is fed to said formaldehyde concentration unit as a first side feed and stream C1 is fed to said formaldehyde concentration unit as a second side feed.
